(19) United States
(12) Patent Application Publication
(10) Pub. No.: US 2010/0255001 A1
(43) Pub. Date: Oct. 7, 2010

(54) BLOCKERS OF SEROTONIN AND ITS RECEPTORS FOR THE TREATMENT OF HEPATITIS

(75) Inventors: Karl Lang, Tubingen (DE); Philipp Lang, Tubingen (DE); Hans Hengartner, Langnau a.A. (CH); Rolf Zinkernagel, Zumikon (CH); Panco Georgiev, Zurich (CH)

Correspondence Address: CONNOLLY BOVE LODGE & HUTZ LLP, 1875 EYE STREET, N.W., SUITE 1100, WASHINGTON, DC, 20006, US

(73) Assignee: Universität Zürich Prorektorat Mnw

(21) Appl. No.: 12/679,732

(22) PCT Filed: Sep. 22, 2008
(86) PCT No.: PCT/EP08/07974

§ 371 Date: Jun. 24, 2010

(30) Foreign Application Priority Data
Sep. 24, 2007 (EP) 07018715.8

(51) Int. Cl. A61K 39/42; A61K 31/138; A61K 31/277; A61K 31/137; A61K 31/4525; A61P 31/22; G01N 33/53

(52) U.S. Cl. 424/161.1; 514/652; 514/523; 514/647; 514/321; 436/501

(57) ABSTRACT

The present invention relates to a method of treating hepatitis comprising administering a serotonin blocker, and the use of such blockers in said treatment and in the manufacture of medicaments for treating hepatitis.

### FIELD OF THE INVENTION

This invention relates to the treatment of hepatitis, particularly virus induced hepatitis, using blockers of serotonin.

### BACKGROUND OF THE INVENTION

Serotonin is a biogenic amine which is an important neurotransmitter in the central nervous system. Besides the production of serotonin in the CNS, serotonin is produced in enterochromaffin cells in the gut. After secretion of serotonin by enterochromaffin cells it is taken up by platelets via the serotonin transporter and is stored in platelet granules in high concentrations. Release of serotonin by platelets is involved in formation of stable clotts and regulation of vasotonus.

This invention focuses on the treatment of hepatitis, a disease induced by autoimmune processes (autoimmune hepatitis, primary biliary cirrhosis), by alcoholic or toxic liver destruction and by infections, particularly by the hepatitis viruses HAV, HBV, HCV, HDV, HEV, and HGV. For hepatitis caused by infections alone around 500 million people worldwide are affected with persistent infection. The standard therapy for hepatitis C is treatment with interferon for two years. Beside a breakup of the therapy due to strong side effects, the clearance of virus is only able in 50% of the cases.

### SUMMARY OF THE INVENTION

The present invention relates to a method of treating hepatitis, particularly virus induced hepatitis, comprising administering a serotonin blocker, and the use of such blockers in said treatment and in the manufacture of medicaments for treating hepatitis.

The invention further relates to a method of screening for a compound effective in the treatment of hepatitis, comprising contacting a candidate compound with serotonin, a serotonin-receptor or a serotonin transporter and choosing candidate compounds which selectively reduce activity of serotonin, the serotonin receptor or the serotonin transporter. The invention further relates to compounds selected by these methods of screening.

### BRIEF DESCRIPTION OF THE FIGURES

FIG. 1:

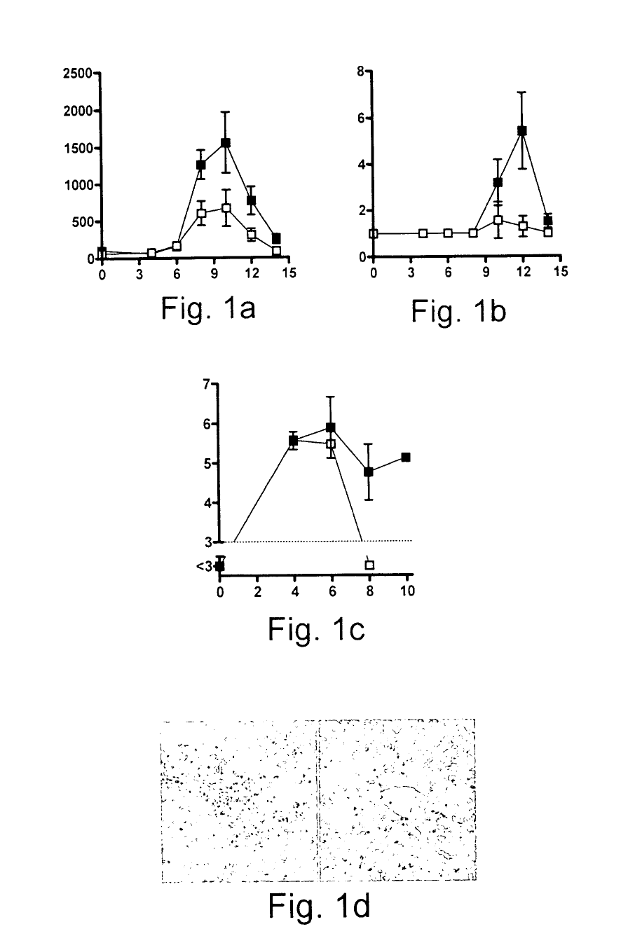

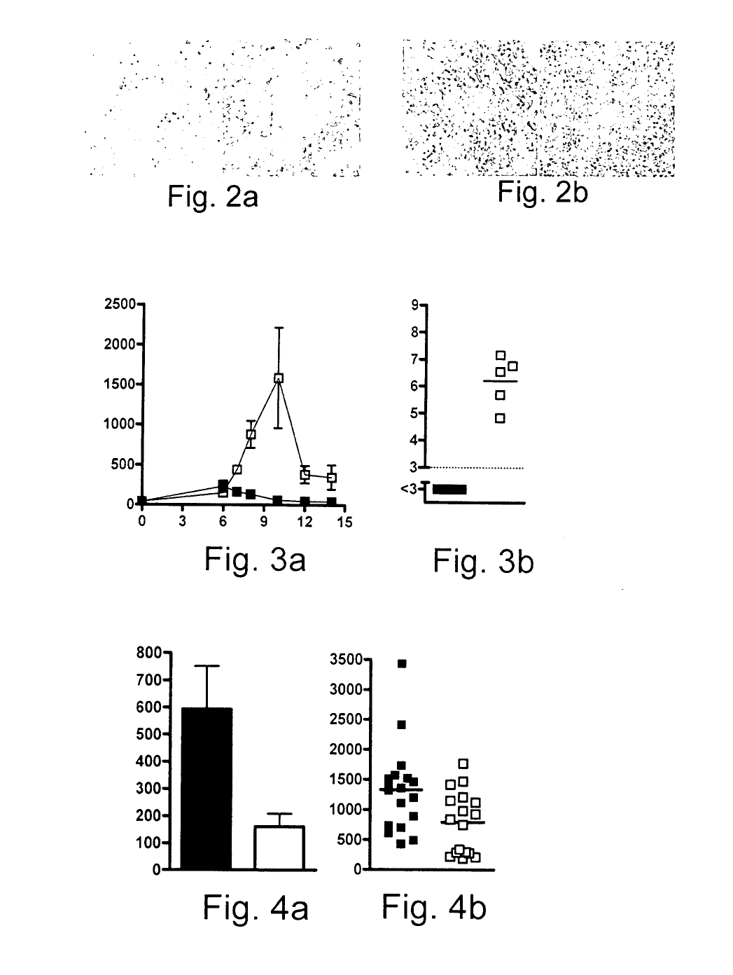

Mice deficient for the rate limiting enzyme for production of serotonin (tryptophanhydrolase-1, tph1) and corresponding control mice were infected with 2×10⁶ pfu LCMV. FIG. 1a: Serum alanine transaminase (ALT) activity was analyzed after LCMV infection of tph1^{-/-} (open symbol) and control mice (closed symbol, n=9, p=0.01); x-axis represents time (days). FIG. 1b: Serum bilirubin concentration were analyzed after LCMV infection of tph1^{-/-} (open symbol) and control mice (closed symbol, n=4, p=0.036); x-axis represents time (days). FIG. 1c: Viral titers in livers of tph1^{-/-} (open symbol) and control mice (closed symbol) were analyzed in plaque assay (n=4); x-axis represents time (days). FIG. 1d: Control mice (left panel, n=3) and tph1 mice (right panel, n=3) were analyzed in histology day 12 after LCMV infection.

FIG. 2:

Neonatal mice deficient for the rate limiting enzyme for production of serotonin (tryptophanhydrolase-1, tph1) and corresponding control mice were infected with 2×10⁴ pfu LCMV-WE. FIG. 2a: After 14 days livers of control mice (left panel, n=5) and livers of tph1^{-/-} mice (right panel, n=7) were analyzed in histology for presence of virus (VL4 staining). FIG. 2b: After 14 days livers of control mice (left panel, n=5) and livers of tph1^{-/-} mice (right panel, n=7) were analyzed in histology for liver cell damage (HE staining).

FIG. 3:

C57BU6 mice were infected with 2×10⁴ pfu of LCMV-WE. One group of mice was further treated with serotonin (5 mg/mouse/day). FIG. 3a: Serotonin treated mice (open symbols) and control mice (closed symbols) were analyzed for ALT activities in the serum; x-axis represents time (days). FIG. 3b: After 8 days livers of serotonin treated mice (open symbols) and control mice (closed symbols) were analyzed for viral titers.

FIG. 4:

C57BU6 mice were infected with 2×10⁶ pfu of LCMV-WE. One group of mice was further treated with fluoxetine (5mg/mouse/day). FIG. 4a: Fluoxetine treated mice (open symbols) and control mice (closed symbols) were analyzed for serotonin in plasma (n=8-10, p=0.03). FIG. 4b: Fluoxetine treated mice (open symbols) and control mice (closed symbols) analyzed for ALT activities in the serum (p=0.017).

### DETAILED DESCRIPTION OF THE INVENTION

The present invention relates to a method of treating hepatitis, particularly virus induced hepatitis, comprising administering a serotonin blocker, and the use of such blockers in said treatment and in the manufacture of medicaments for treating hepatitis.

Hepatitis can be induced by autoimmune processes (autoimmune hepatitis, primary biliary cirrhosis), by alcoholic liver destruction and by infections, particularly the hepatitis viruses HAV, HBV, HCV, HDV, HEV, and HGV.

The action of serotonin can be inhibited in several ways. The production can be inhibited by trypthophan hydrolase inhibitors (see list below). The uptake of serotonin by platelets or other target organs can be inhibited by serotonin reuptake inhibitors (see list below). The storage and the release of serotonin can be inhibited (see list below). The action of serotonin can be inhibited by serotonin receptor blockers. The receptors for serotonin are under permanent transcriptional control. Therefore a strong agonist leads to downregulation of receptors or their activity which leads to a “paradoxically” reduced serotonin signalling (see list below). The degradation of serotonin can be inhibited by monoamine oxidase inhibitors (see list below). Targeting of the serotonin pathway can be achieved by the administration of neutralizing antibodies or antibody fragments to serotonin or serotonin receptors or by the therapeutic use of proteins or synthetic compounds (such as repeat proteins as described in WO 02/20565), which bind serotonin, and thereby prevent its binding to serotonin receptors or bind to a serotonin receptor (see list below).

Further examples of serotonin blockers according to the invention are compounds which by binding to serotonin interfere with serotonin receptor activation (see below). On the level of the membrane receptor, serotonin binding can be inhibited with soluble serotonin receptors.

Serotonin blockers according to the invention are disclosed in the following. However, the invention is not restricted to the serotonin blockers disclosed therein, but extends to all serotonin blockers.

Preferred serotonin blockers according to the invention are:

A: Inhibitor of serotonin production (trypthophan hydrolase inhibitors):

p-chlorophenylalaninine

B: Inhibitior of serotonin storage: Reserpine (SIGMA)

C: Molecules effecting release of serotonin: Fenfluramine (SIGMA), MDMA (SIGMA), p-chloroamphetamine (SIGMA), Parthenolide (SIGMA)

D: Receptor antagonists: NAN 190 (SIGMA), Spiroxatrine (SIGMA), WAY 100135(Institute of Pharmacology, Polish Academy of Sciences, Krakow, Poland; Komabiotech, Korea), Pindolol (Komabiotech, Korea), Isamoltane (Komabiotech, Korea), BRL 15572 (SIGMA and Komabiotech, Korea), GR 55562 (SIGMA), AltanserineCyproheptadine (SIGMA), Cinanserin, Metergoline (SIGMA), Methysergide (SIGMA), Ritanserin (SIGMA), Sulpiride (SIGMA), Ketanserin (SIGMA), Mianserin (SIGMA), Spiperone (SIGMA), MDL-72222 (SIGMA), Zacopride (Sanofi-Aventis, France), Ondansetron (SIGMA), ICS 205-930 (SIGMA), GR113808 (Janssen Research Foundation), RS 39604 (Br J Pharmacol. 1995 July; 115 (6):1087-95), Clozapine (SIGMA), SB 2699710, Cyanopindolol hemifumarate (Komabiotech, Korea), GR 127935 hydrochloride (Komabiotech, Korea), MM 77 dihydrochloride (Komabiotech, Korea), NAN-190 hydrobromide (Komabiotech, Korea), NAS-181 (Komabiotech, Korea), SB 216641 (Komabiotech, Korea), SB 224289 (Komabiotech, Korea), Spiroxatrine (Komabiotech, Korea).

E: Receptor agonists, leading to downregulation of receptors or their activity (all from Komabiotech, Korea): Anpirtoline hydrochloride, BMY 7378 dihydrochloride, BP-554 maleate, BRL 54443, Buspirone hydrochloride, 5-Carboxamidotryptamine maleate, CGS-12066B dimaleate, CP 93129 dihydrochloride, CP 94253 hydrochloride, Eltoprazine hydrochloride, GR 46611, 8-Hydroxy-DPAT hydrobromide, (R)-(+)-8-Hydroxy DPAT hydrobromide, 8-Hydroxy-PIPAT, Ipsapirone, L-694,247, MDL 73005EF hydrochloride, 5-Nonyloxytryptamine oxalate, RU 24969 hemisuccinate, S 14506 hydrochloride, TFMPP hydrochloride, Urapidil hydrochloride

F: Reuptake blockers: Clomipramine (SIGMA), Citolopram (Celexa), Fenfluramine (SIGMA), Fluoextine (Eli Lilly), Fluvoxamine (SIGMA), Indatraline (SIGMA), Imipramine (SIGMA), Quipazine (SIGMA), Paroxetine (GlaxoSmithKline, SIGMA), Roxindole (Merck), Setraline (Pfizer), Trazadone (Teva), Zimelidine (SIGMA), escitalopram (Lundbeck), dapoxetine (ALZA).

G: Monoamine oxidase inhibitors: Deprenyl (SIGMA), Harmane (SIGMA), Paragline, Cloglyline, Tranylcypromine (SIGMA), Nialamide (SIGMA), Ipronoziazid, Tetrindole ((2,3,3a,4,5,6-hexahydro-8-cyclohexyl-1H[3,2,1-j,k] carbazole).

H: Depletion of peripheral serotonin by antibodies or antigen binding fragments of an antibody (e.g. Fab fragments): mouse anti serotonin (MA2027,cell science), mouse anti-Serotonin (Clone 5HT-H209, RDI-SEROTabm-29, RDI, Research Diagnostics) and mouse anti-serotonin (53842, Ana-Spec).

Most preferred serotonin blockers according to the invention are fluoxetine (SIGMA), fluvoxamine (Luvox, SIGMA), sertraline (Pfizer) paroxetine (GlaxoSmithKline, SIGMA), serotonin and serotonin receptor antibodies or antigen binding fragments of an antibody, or antibody like molecules (repeat proteins binding to serotonin or serotonin receptors).

One aspect of the invention relates to a method of treating hepatitis, particularly virus induced hepatitis, comprising administering serotonin blockers as defined hereinbefore in a quantity effective against hepatitis to a mammal in need thereof, for example to a human requiring such treatment. The treatment may be for prophylactic or therapeutic purposes. For the administration, the serotonin blocker is preferably in the form of a pharmaceutical preparation comprising the serotonin blocker in chemically pure form and optionally a pharmaceutically acceptable carrier and optionally adjuvants. The serotonin blocker is used in an amount effective against hepatitis. The dosage of the active ingredient depends upon the species, its age, weight, and individual condition, the individual pharmacokinetic data, the mode of administration, and whether the administration is for prophylactic or therapeutic purposes. In the case of an individual having a bodyweight of about 70 kg the daily dose administered is from approximately 1 mg to approximately 1000 mg, preferably from approximately 50 mg to approximately 1000 mg, of a serotonin blocker.

Pharmaceutical compositions for enteral administration, such as nasal, buccal, rectal or, especially, oral administration, and for parenteral administration, such as subcutaneous, intravenous, or intramuscular are especially preferred. The pharmaceutical compositions comprise from approximately 1% to approximately 95% active ingredient, preferably from approximately 20% to approximately 90% active ingredient.

For parenteral administration preference is given to the use of solutions of the serotonin blockers, and also suspensions or dispersions, especially isotonic aqueous solutions, dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

Transdermal application is also considered, for example using a transdermal patch, which allows administration over an extended period of time, e.g. from one to twenty days.

Another aspect of the invention relates to the use of serotonin blockers as described hereinbefore in the treatment of hepatitis, particularly virus induced hepatitis, and in the manufacture of medicaments for treating this disease. Such medicaments are manufactured by methods known in the art, especially by conventional mixing, coating, granulating, dissolving or lyophilizing.

The serotonin blocker can be administered alone or in combination with one or more other therapeutic agents, possible combination therapy taking the form of fixed combinations of a serotonin blocker and one or more other therapeutic agents known in the treatment of hepatitis, the administration being staggered or given independently of one another, or being in the form of a fixed combination.

Possible combination partners considered are anti-infective compounds (in the event of a hepatitis caused by an infection) or anti-inflammatory compounds.

The invention further relates to a method of screening for a compound effective in the treatment of hepatitis comprising contacting a candidate compound with serotonin or a serotonin receptor and choosing candidate compounds which selectively reduce the activity of serotonin. The invention further relates to compounds selected by these methods of screening.

Blockers of serotonin activity are identified by contacting serotonin or a serotonin receptor with a candidate compound. A control assay with the corresponding serotonin or serotonin receptor—in the absence of the candidate compound—is run in parallel. A decrease in activity in the presence of the candidate compound compared to the level in the absence of the compound indicates that the compound is a serotonin blocker.

Antibodies against serotonin can be e.g. be generated in serotonin knock-out mice (serotonin−/− mouse) by immunizing such mice with serotonin and adjuvants. B cells of such mice are then fused according to standard protocols. Efficiency of inhibiting the activity of serotonin can be screened by ELISA assays.

### Concepts And Evidence Behind the Invention

To analyze the role of serotonin in hepatitis tryptophan hydrolase-1-deficient (tph1−/−) mice were infected with LCMV. In these mice, the formation of serotonin is blocked due to the absence of this critical enzyme. The serum alanine transaminase (ALT) activity and serum bilirubin concentrations, which both correlate directly with liver cell damage, were significantly reduced in LCMV infected tph1−/− mice (FIG. 1a and b), correlating with a limited hepatitis as assessed by histology (FIG. 1d). While early replication of virus in the liver was comparable in tph1−/− and control mice, CD8⁺ T cell mediated virus elimination below detection level was faster in tph1−/− livers (FIG. 1c). This was unexpected and no apparent difference in CD8⁺ T cell infiltrates between tph1−/− and control mice could be demonstrated.

To analyze the role of serotonin in general hepatitis, neonatal mice deficient for the rate limiting enzyme for production of serotonin (tryptophanhydrolase-1, tph1) and corresponding control mice were infected with 2×10⁴ pfu LCMV-WE. After 14 days livers of control mice and livers of tph1^{-/-} mice were analyzed for pathological changes. Both mice were not able to control the virus (FIG. 2a). Tph1^{-/-} mice showed however limited immunopathology (FIG. 2b) which proves to involvement of serotonin in hepatitis as such.

To assess whether serotonin directly influences hepatitis in the liver C57BL/6 mice were treated with exogenous serotonin and infected with an intermediate dose of LCMV, which normally induces a very mild hepatitis (2×10⁴ pfu). In vehicle treated mice, LCMV was cleared by day 8 and ALT levels in the serum showed only a slight raise around day 6 (FIG. 3a). In mice treated with serotonin high levels of replicating virus was found eight days after infection (FIG. 3b). ALT levels reached more than 1000 U/I with a maximum at day 10 after infection (FIG. 3a). Serotonin treatment without infection was harmless to the liver. Furthermore LCMV infected mice were treated with a serotonin re-uptake inhibitor and the ALT activity was measured on day 10. A significant decrease in ALT levels was found.

To assess the role of the serotonin re-uptake inhibitor fluoxetine, C57BL/6 mice were infected with 2×10⁶ pfu of LCMV-WE. One group of mice was further treated with fluoxetine (5 mg/mouse/day). Fluoxetine treated mice had reduced serotonin in the plasma (FIG. 4a) and showed significantly reduced damage of hepatocytes compared to control mice (FIG. 4b).

1. A method of treating hepatitis comprising administering a serotonin blocker in an effective amount to a patient in need thereof.

2. The method of claim 1 for treating virus induced hepatitis.

3. The method according to claim 1, wherein the serotonin blocker is a compound which inhibits serotonin production, storage, or release of serotonin, which acts as a serotonin receptor antagonist, which acts as a serotonin receptor agonist leading to downregulation of receptors or their activity, which is a serotonin reuptake inhibitor, a monoamine oxidase inhibitor, or leads to depletion of peripheral serotonin.

4. The method according to claim 1, wherein the TGFβ blocker is selected from the group consisting of antibodies or antigen binding fragments of an antibody to serotonin and serotonin receptors, repeat proteins to serotonin and serotonin receptors, fluoxetine, fluvoxamine, sertraline, and paroxetine.

5. (canceled)

6. A method of treating virus induced hepatitis comprising administering a serotonin blocker in an effective amount to a patient in need thereof.

7. A method of screening for a compound effective in the treatment of hepatitis comprising contacting a candidate compound with serotonin or a serotonin receptor, and choosing candidate compounds which selectively reduce activity of serotonin or the serotonin receptor.

8. A compound selected by the method of claim 7.

9. The method according to claim 2, wherein the serotonin blocker is a compound which inhibits serotonin production, storage, or release of serotonin, which acts as a serotonin receptor antagonist, which acts as a serotonin receptor agonist leading to downregulation of receptors or their activity, which is a serotonin reuptake inhibitor, a monoamine oxidase inhibitor, or leads to depletion of peripheral serotonin.

10. The method according to claim 2, wherein the TGFβ blocker is selected from the group consisting of antibodies or antigen binding fragments of an antibody to serotonin and serotonin receptors, repeat proteins to serotonin and serotonin receptors, fluoxetine, fluvoxamine, sertraline, and paroxetine
